(19) United States
(12) Patent Application Publication
(10) Pub. No.: US 2008/0300290 A1
(43) Pub. Date: Dec. 4, 2008

(54) Novel Beta-Agonists, Process for Their Preparation and Their Use as Medicaments

(76) Inventors: Rainer Walter, Biberach (DE); Thomas Trieselmann, Warthausen (DE); Matthew R. Netherton, Danbury, CT (US); Marco Santagostino, Mittelbiberach (DE); Bradford S. Hamilton, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 12/091,823

(22) PCT Filed: Oct. 27, 2006
(86) PCT No.: PCT/EP2006/067868

§ 371 Date: Apr. 28, 2008

(30) Foreign Application Priority Data
Oct. 28, 2005 (DE) 102005052101.0

(51) Int. Cl. A61K 31/4164; C07D 233/61; A61P 3/04; A61P 3/10

(52) U.S. Cl. 514/399; 548/340.1

(57) ABSTRACT

Compounds of general formula (I)

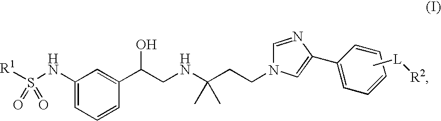

which are selective beta-3-agonists and useful for the treatment of obesity and type II diabetes. Exemplary compounds are:
[4-(1-{3-[2-(3-phenylsulphonylamino-phenyl)-2-hydroxy-ethylamino]-3-methyl-butyl}-1H-imidazol-4-yl)-phenyl]-acetic acid and
methyl [4-(1-{3-[2-(3-phenylsulphonylamino-phenyl)-2-hydroxy-ethylamino]-3-methyl-butyl}-1H-imidazol-4-yl)-phenyl]-acetate.

The present invention relates to new beta-agonists of general formula (I)

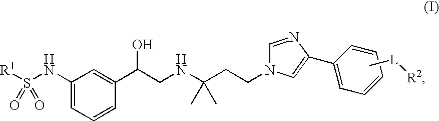

wherein the groups L, R¹ and R² have the meanings given in the claims and specification, the tautomers, racemates, enantiomers, diastereomers, solvates, hydrates thereof, mixtures thereof and the salts thereof, particularly the physiologically acceptable salts thereof with inorganic or organic acids or bases, processes for preparing these compounds and their use as medicaments.

### BACKGROUND TO THE INVENTION

The treatment of type II diabetes and obesity is based primarily on reducing calorie intake and increasing physical activity. These methods are seldom successful in the long term.

It is known that beta-3 receptor agonists exhibit a significant effect on lipolysis, thermogenesis and the serum glucose level in animal models of type II diabetes (Arch JR. beta(3)-Adrenoceptor agonists: potential, pitfalls and progress, Eur J Pharmacol. 2002 Apr. 12; 440(2-3):99-107).

Compounds that are structurally similar to the compounds according to the invention and their broncholytic, spasmolytic and antiallergic activity were disclosed for example in DE 2833140.

The aim of the present invention is to provide selective beta-3-agonists which are suitable for preparing medicaments for the treatment of obesity and type II diabetes.

### DETAILED DESCRIPTION OF THE INVENTION

Surprisingly it has been found that compounds of general formula (I) wherein the groups L, R¹ and R² have the meanings given below act as selective beta-3-agonists. Thus the compounds according to the invention may be used for the treatment of ailments connected with the stimulation of beta-3-receptors.

The present invention therefore relates to compounds of general formula (I)

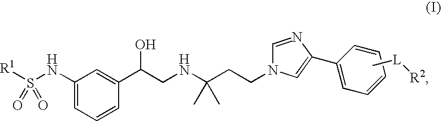

wherein
R¹ denotes a phenyl group which may be substituted by one to three fluorine, chlorine or bromine atoms or one to three C₁₋₃-alkyl, C₁₋₃-alkyloxy, trifluoromethoxy or difluoromethoxy groups, wherein the substituents may be identical or different,
L denotes a C₁₋₃-alkylene group wherein a methylene group may be replaced by an oxygen or sulphur atom or by a NH group, while L may be substituted in the alkyl moiety by one or two methyl groups, and
R² denotes a carboxy or C₁₋₃-alkoxy-carbonyl group,
while the alkyl groups contained in the above-mentioned groups may each be straight-chain or branched,
and the tautomers, racemates, enantiomers, diastereomers, solvates, hydrates, mixtures thereof and salts thereof, and optionally the prodrugs, double prodrugs and salts thereof, particularly the physiologically acceptable salts thereof with inorganic or organic acids or bases.

Preferred compounds of general formula (I) are those wherein

R² is as hereinbefore defined,
R¹ denotes a phenyl group which may be substituted by a fluorine, chlorine or bromine atom or a C₁₋₃-alkyl, C₁₋₃-alkyloxy, trifluoromethoxy or difluoromethoxy group, and
L denotes a C₁₋₃-alkylene group or a —O—CH₂— group, wherein L may be substituted in the alkyl moiety by one or two methyl groups,
the tautomers, the enantiomers, the diastereomers, the mixtures thereof and the salts thereof.

Particularly preferred are compounds of general formula (I), wherein

R² is as hereinbefore defined,
R¹ denotes a phenyl group and
L denotes a —CH₂, —CH₂—CH₂, —O—CH₂ or —O—C(CH₃)₂— group,
the tautomers, the enantiomers, the diastereomers, the mixtures thereof and the salts thereof.

A preferred sub-group relates to those compounds of general formula (I), wherein the groups L, R¹ and R² are as hereinbefore defined, wherein the group -L-R² is in position 3 or 4 of the phenyl ring, particularly those compounds of general formula (I), wherein the group -L-R² is in the 4 position of the phenyl ring.

Another preferred sub-group relates to the (R)-enantiomer of formula (Ia)

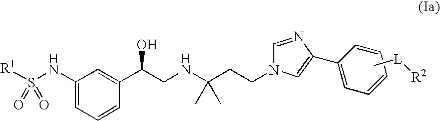

of the compounds according to the invention, wherein the groups L, R¹ and R² are as hereinbefore defined, and the salts thereof.

A third preferred sub-group relates to the (S)-enantiomer of formula (Ib)

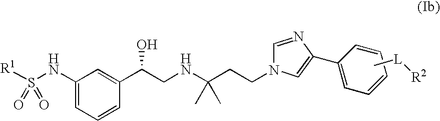

of the compounds according to the invention, wherein the groups L, R¹ and R² are as hereinbefore defined, and the salts thereof.

Particularly preferred are the following compounds:
[4-(1-{3-[2-(3-phenylsulphonylamino-phenyl)-2-hydroxy-ethylamino]-3-methyl-butyl}-1H-imidazol-4-yl)-phenyl]-acetic acid,
methyl [4-(1-{3-[2-(3-phenylsulphonylamino-phenyl)-2-hydroxy-ethylamino]-3-methyl-butyl}-1H-imidazol-4-yl)-phenyl]-acetate,
[3-(1-{3-[2-(3-phenylsulphonylamino-phenyl)-2-hydroxy-ethylamino]-3-methyl-butyl}-1H-imidazol-4-yl)-phenyl]-acetic acid,
[4-(1-{3-[2-(3-phenylsulphonylamino-phenyl)-2-hydroxy-ethylamino]-3-methyl-butyl}-1H-imidazol-4-yl)-phenoxy]-acetic acid,
3-[4-(1-{3-[2-(3-phenylsulphonylamino-phenyl)-2-hydroxy-ethylamino]-3-methyl-butyl}-1H-imidazol-4-yl)-phenyl]-propionic acid,
3-[3-(1-{3-[2-(3-phenylsulphonylamino-phenyl)-2-hydroxy-ethylamino]-3-methyl-butyl}-1H-imidazol-4-yl)-phenyl]-propionic acid,
ethyl 3-[3-(1-{3-[2-(3-phenylsulphonylamino-phenyl)-2-hydroxy-ethylamino]-3-methyl-butyl}-1H-imidazol-4-yl)-phenyl]-propionate,
[3-(1-{3-[2-[3-(phenylsulphonylamino)-phenyl]-2-hydroxy-ethylamino]-3-methyl-butyl}-1H-imidazol-4-yl)-phenoxy]-acetate ethyl,
[3-(1-{3-[2-[3-(phenylsulphonylamino)-phenyl]-2-hydroxy-ethylamino]-3-methyl-butyl}-1H-imidazol-4-yl)-phenoxy]-acetic acid and
2-[4-(1-{3-[(R)-2-[3-(phenylsulphonylamino)-phenyl]-2-hydroxy-ethylamino]-3-methyl-butyl}-1H-imidazol-4-yl)-phenoxy]-2-methyl-propionic acid
   and the enantiomers and salts thereof.

The invention further relates to compounds of general formula (I) for use as pharmaceutical compositions.

The invention further relates to compounds of general formula (I) for use as pharmaceutical compositions with a selective beta-3-agonistic activity.

The invention further relates to compounds of general formula (I) for preparing a pharmaceutical composition for the treatment and/or prevention of diseases which are associated with the stimulation of beta-3-receptors.

The invention further relates to a method for the treatment and/or prevention of diseases which are associated with the stimulation of beta-3-receptors, by administering to a patient an effective amount of a compound of general formula I.

The invention further relates to a pharmaceutical composition, containing as active substance one or more compounds of general formula (I) optionally in combination with conventional excipients and/or carriers.

The invention further relates to a pharmaceutical composition containing as active substance one or more compounds of general formula (I) or the physiologically acceptable salts thereof and one or more active substances selected from among the antidiabetic agents, inhibitors of protein tyrosine phosphatase 1, substances that influence deregulated glucose production in the liver, lipid lowering agents, cholesterol absorption inhibitors, HDL-raising compounds, active substances for the treatment of obesity and modulators or stimulators of the adrenergic system through alpha 1 and alpha 2 as well as beta 1, beta 2 and beta 3 receptors.

The invention further relates to a process for preparing a compound of general formula (I)

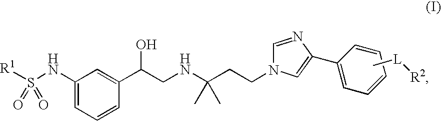

wherein
a) in order to prepare a compound of general formula (I) wherein
L, R¹ and R² may have the meanings given above,
a compound of formula (II)

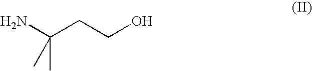

is converted, using a chlorinating agent, into a compound of formula (III)

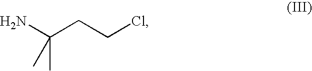

the compound of formula (III), optionally provided with an amino protective group, is converted with 4-iodoimidazole into a compound of formula (IV),

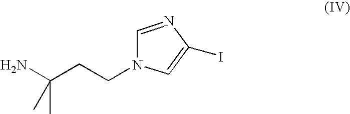

the compound of formula (IV) is reacted with a compound of formula (V)

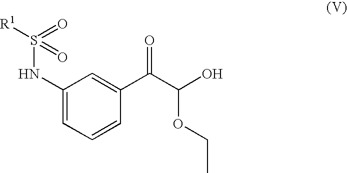

wherein R¹ has the meaning given above,
and the compound thus obtained of formula (VI)

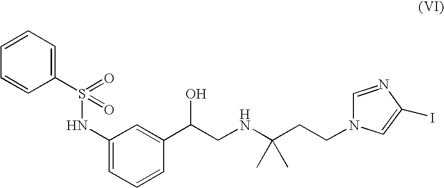

is coupled with a compound of general formula (VII),

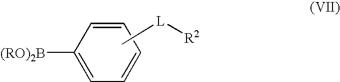

wherein L and R² have the meaning given above and the groups R each independently of one another denote a hydrogen atom or a straight-chain or branched C₁₋₄-alkyl group or together may denote an optionally branched C₂₋₆-alkylene group,
and then if desired separation of the enantiomers is carried out and/or optionally the compound of formula I thus obtained is converted into one of the salts thereof; or
b) in order to prepare a compound of general formula (I) wherein
R¹ and R² may have the meanings given above and
L denotes a C₁₋₃-alkylene group wherein the methylene group linked to the phenyl ring is replaced by an oxygen or sulphur atom, while L may be substituted in the alkyl moiety by one or two methyl groups,
wherein a compound of formula (VIII)

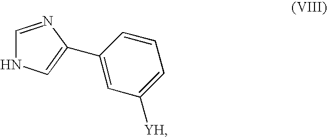

wherein Y denotes an oxygen or sulphur atom,
is converted, using a suitable protective group for the NH group (for example a tert-butyloxycarbonyl or a triphenylmethyl group) by reaction with a compound of formula

R²′-X,

wherein R²′ is identical to a group R² as hereinbefore defined or denotes a group that can be converted into a group R² as hereinbefore defined and
X denotes a suitable leaving group, such as e.g. a halogen atom or a C₁₋₃-alkylsulphonyloxy, trifluoromethylsulphonyloxy or arylsulphonyloxy group, or a hydroxy group,
into a compound of general formula (IX)

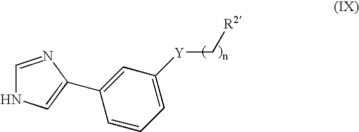

wherein R²′ and Y are as hereinbefore defined and n denotes a number selected from among 0, 1 and 2,
the compound of formula (IX) is converted with a compound of formula (X), (XI) or (XII)

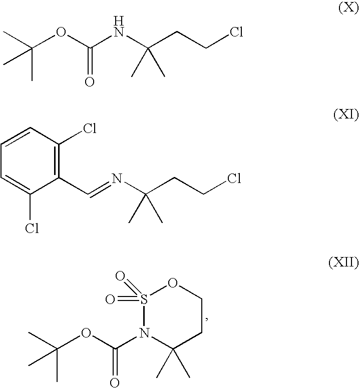

into a compound of general formula (XIII),

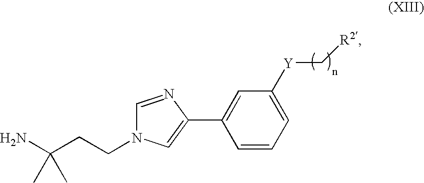

wherein Y, n and R²′ are as hereinbefore defined, and the group R²′ is optionally converted into the group R² simultaneously or subsequently, possibly by transesterification,
and the compound of general formula (XIII) is reacted with a compound of general formula (XIVa) or (XIVb)

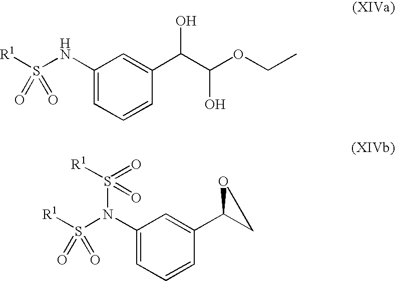

wherein R¹ in each case has the meaning given above,
in order to obtain the compound of general formula (I) wherein L denotes a C₁₋₃-alkylene group wherein the methylene group linked to the phenyl ring is replaced by an oxygen or sulphur atom, wherein L may be substituted in the alkyl moiety by one or two methyl groups,
and subsequently, if desired, desulphonation and/or separation of enantiomers is carried out and/or the compound of general formula (I) thus obtained is optionally converted into one of the salts thereof.

The reaction with the compound (XIVa) leads to the racemate, whereas the synthesis with the compound (XIVb) yields the respective (R)-enantiomer. An analogous reaction with the enantiomer of (XIVb), leading to the (S)-enantiomer, is naturally also possible.

By alkyl groups, as well as alkyl groups, which are a part of other groups, are meant, unless stated otherwise, branched and unbranched alkyl groups with 1 to 10 carbon atoms, while groups with 1 to 6 carbon atoms are preferred. Particularly preferred are alkyl groups with 1 to 4 carbon atoms, particularly those with 1 or 2 carbon atoms. Examples include: methyl, ethyl, propyl, butyl, pentyl, hexyl, heptyl, octyl, nonyl and decyl. Unless stated otherwise, the above-mentioned terms propyl, butyl, pentyl, hexyl, heptyl, octyl, nonyl and decyl include all the possible isomeric forms. For example the term propyl includes the two isomeric groups n-propyl and iso-propyl, the term butyl includes n-butyl, iso-butyl, sec. butyl and tert.-butyl, the term pentyl includes isopentyl, neopentyl etc.

In the above-mentioned alkyl groups one or more hydrogen atoms may optionally be replaced by other groups. For example these alkyl groups may be substituted by the halogen atoms fluorine, chlorine, bromine or iodine. The substituents are preferably fluorine or chlorine. The substituent fluorine is particularly preferred. If desired all the hydrogen atoms of the alkyl group may be replaced.

Similarly, in the above-mentioned alkyl groups, unless stated otherwise, one or more hydrogen atoms may optionally be replaced for example by OH, NO₂, CN or an optionally substituted group selected from among —O—(C₁-C₅-alkyl), preferably methoxy or ethoxy, —O—(C₆-C₁₄-aryl), preferably phenyloxy, —O-heteroaryl, preferably —O-thienyl, —O-thiazolyl, —O-imidazolyl, —O-pyridyl, —O-pyrimidyl or —O-pyrazinyl, saturated or unsaturated —O-heterocycloalkyl, preferably —O-pyrazolyl, —O-pyrrolidinyl, —O-piperidinyl, —O-piperazinyl or —O-tetrahydro-oxazinyl, C₆-C₁₄-aryl, preferably phenyl, heteroaryl, preferably thienyl, thiazolyl, imidazolyl, pyridyl, pyrimidyl or pyrazinyl, saturated or unsaturated heterocycloalkyl, preferably pyrazolyl, pyrrolidinyl, piperidinyl, piperazinyl or tetrahydro-oxazinyl, an amine group, preferably methylamine, benzylamine, phenylamine or heteroarylamine, saturated or unsaturated bicyclic ring systems, preferably benzimidazolyl and C₃-C₈-cycloalkyl, preferably cyclohexyl or cyclopropyl.

By alkenyl groups, as well as alkenyl groups which are a part of other groups, are meant branched and unbranched alkyl groups with 1 to 10 carbon atoms, preferably 1 to 6, particularly preferably 1 to 4 carbon atoms, which contain at least one carbon-carbon double bond. Examples include: ethenyl, propenyl, methylpropenyl, butenyl, pentenyl, hexenyl, heptenyl, methylheptenyl, octenyl, nonenyl and decenyl. Unless stated otherwise, the above-mentioned terms propenyl, butenyl, pentenyl, hexenyl, heptenyl, octenyl, nonenyl and decenyl include all the possible isomeric forms. For example the term butenyl includes the isomeric groups but-1-enyl, but-2-enyl and but-3-enyl, etc.

In the above-mentioned alkenyl groups one or more hydrogen atoms may optionally be replaced by other groups. For example these alkenyl groups may be substituted by the halogen atoms fluorine, chlorine, bromine or iodine. The substituents fluorine or chlorine are preferred. The substituent fluorine is particularly preferred. If desired, all the hydrogen atoms of the alkenyl group may optionally also be replaced.

By alkynyl groups, as well as alkynyl groups which are a part of other groups, are meant branched and unbranched alkyl groups with 1 to 10 carbon atoms, preferably 1 to 6, particularly preferably 1 to 4 carbon atoms, which contain at least one carbon-carbon triple bond. Examples include: ethynyl, propynyl, butynyl, pentynyl, hexynyl, heptynyl, octynyl, nonynyl and decynyl. Unless stated otherwise, the above-mentioned terms propynyl, butynyl, pentynyl, hexynyl, heptynyl, octynyl, nonynyl and decynyl include all the possible isomeric forms. For example the term butynyl includes the isomeric groups but-1-ynyl, but-2-ynyl and but-3-ynyl, etc.

In the above-mentioned alkynyl groups one or more hydrogen atoms may optionally be replaced by other groups. For example these alkynyl groups may be substituted by the halogen atoms fluorine, chlorine, bromine or iodine. The substituents fluorine or chlorine are preferred. The substituent fluorine is particularly preferred. If desired, all the hydrogen atoms of the alkynyl group may optionally also be replaced.

The term aryl denotes an aromatic ring system with 6 to 18 carbon atoms, preferably 6 to 14 carbon atoms, preferably 6 or 10 carbon atoms, particularly preferably phenyl, which may optionally be substituted and may preferably carry one or more of the following substituents: OH, NO₂, CN, —OCHF₂, —OCF₃, —NH₂, —NH-alkyl, —N(alkyl)-alkyl, —NH-aryl, —N(alkyl)-aryl, —NHCO-alkyl, —NHCO-aryl, —N(alkyl)-CO-alkyl, —N(alkyl)-CO-aryl, —NHSO₂-alkyl, —NHSO₂—N(alkyl)₂, —NHSO₂-aryl, —N(alkyl)-SO₂-alkyl, —N(alkyl)-SO₂-aryl, —CO₂-alkyl, —SO₂-alkyl, —SO₂-aryl, —CONH(OH), —CONH-alkyl, —CONH-aryl, —CON(alkyl)-alkyl, —CON(alkyl)-aryl, —SO₂NH-alkyl, —SO₂NH-aryl, —SO₂N(alkyl)-alkyl, —SO₂N(alkyl)-aryl, —O-alkyl, —O-aryl —S-alkyl, —S-aryl, tetrazolyl, halogen, for example fluorine, chlorine, bromine or iodine, preferably fluorine or chlorine, particularly fluorine, C₁-C₁₀-alkyl, preferably C₁-C₅-alkyl, particularly preferably C₁-C₃-alkyl, most particularly preferably methyl or ethyl, —O—(C₁-C₃-alkyl), preferably methoxy or ethoxy, —COOH or —CONH₂.

By heteroaryl groups are meant 5- to 10-membered mono- or bicyclic heteroaryl rings, wherein one to three carbon atoms may be replaced in each case by a heteroatom selected from among oxygen, nitrogen or sulphur. Examples include furan, thiophene, pyrrole, pyrazole, imidazole, triazole, tetrazole, pyridine, pyridazine, pyrimidine, pyrazine, triazine, oxazole, isoxazole, thiazole, thiadiazole, oxadiazole, while each of the above-mentioned heterocycles may optionally also be annelated to a benzene ring, such as for example benzimidazole, and these heterocycles may optionally be substituted and may preferably carry one or more of the following substituents: OH, NO₂, CN, —NH₂, —NH-alkyl, —N(alkyl)-alkyl, —NH-aryl, —N(alkyl)-aryl, —NHCO-alkyl, —NHCO-aryl, —N(alkyl)-CO-alkyl, —N(alkyl)-CO-aryl, —NHSO₂-alkyl, —NHSO₂-aryl, —N(alkyl)-SO₂-alkyl, —N(alkyl)-SO₂-aryl, —CO₂-alkyl, —SO₂-alkyl, —SO₂-aryl, —CONH-alkyl, —CONH-aryl, —CON(alkyl)-alkyl, —CON(alkyl)-aryl, —SO₂NH-alkyl, —SO₂NH-aryl, —SO₂N(alkyl)-alkyl, —SO₂N(alkyl)-aryl, —O-alkyl, —O-aryl —S-alkyl, —S-aryl, —CONH₂, halogen, preferably fluorine or chlorine, C₁-C₁₀-alkyl, preferably C₁-C₅-alkyl, preferably C₁-C₃-alkyl, particularly preferably methyl or ethyl, —O—(C₁-C₃-alkyl), preferably methoxy or ethoxy, —COOH, —COOCH₃, —CONH₂, —SO-alkyl, —SO₂-alkyl, —SO₂H, —SO₃-alkyl or optionally substituted phenyl.

The term cycloalkyl groups denotes saturated or unsaturated cycloalkyl groups with 3 to 8 carbon atoms such as for example cyclopropyl, cyclobutyl, cyclopentyl, cyclo-pentenyl, cyclohexyl, cyclohexenyl, cycloheptyl or cyclooctyl, preferably cyclopropyl, cyclopentyl or cyclohexyl, while each of the above-mentioned cycloalkyl groups may optionally also carry one or more substituents or be annelated to a benzene ring.

By heterocycloalkyl or heterocyclyl groups are meant, unless otherwise described in the definitions, 5-, 6- or 7-membered, saturated or unsaturated heterocycles, which may contain nitrogen, oxygen or sulphur as heteroatoms, such as for example tetrahydrofuran, tetrahydrofuranone, γ-butyrolactone, α-pyran, γ-pyran, dioxolane, tetrahydropyran, dioxane, dihydrothiophene, thiolan, dithiolan, pyrroline, pyrrolidine, pyrazoline, pyrazolidine, imidazoline, imidazolidine, tetrazole, piperidine, pyridazine, pyrimidine, pyrazine, piperazine, triazine, tetrazine, morpholine, thiomorpholine, diazepan, oxazine, tetrahydro-oxazinyl, isothiazole, pyrazolidine, preferably pyrazolyl, pyrrolidinyl, piperidinyl, piperazinyl or tetrahydro-oxazinyl, while the heterocyclic group may optionally be substituted.

The compounds of the above general formula (I) which contain a group that can be cleaved in-vivo are so-called prodrugs, and compounds of general formula I which contain two groups that can be cleaved in-vivo are so-called double prodrugs.

By a group that can be converted in-vivo into a carboxy group is meant for example an ester of formula —CO₂R¹¹, wherein

R¹¹ denotes hydroxymethyl, alkenyl, alkynyl, aryl, heteroaryl, cycloalkenyl, hetero-cycloalkyl, C₁-C₃-alkoxycarbonyl, 1,3-dihydro-3-oxo-1-isobenzofuranol, —C(-alkyl)(-alkyl)-OC(O)-alkyl, —CHC(O)NH(-alkyl), —CHC(O)N(-alkyl)(-alkyl),
alkyl, preferably C₁-C₆-alkyl, particularly preferably methyl, ethyl, n-propyl, iso-propyl, n-butyl, n-pentyl or n-hexyl,
cycloalkyl, preferably C₁-C₆-cycloalkyl, particularly preferably cyclohexyl,
—(C₁-C₃-alkyl)-aryl, preferably (C₁-C₃-alkyl)-phenyl, particularly preferably benzyl,
—CHC(O)N(-alkyl)(-alkyl), preferably —CHC(O)N(—C₁-C₃-alkyl)(—C₁-C₃-alkyl), particularly preferably —CHC(O)N(CH₃)₂,
—CH(-alkyl)OC(O)-alkyl, preferably —CH(—CH₃)OC(O)(—C₁-C₆-alkyl), particularly preferably —CH(—CH₃)OC(O)-methyl, —CH(—CH₃)OC(O)-ethyl, —CH(—CH₃)OC(O)-n-propyl, —CH(—CH₃)OC(O)-n-butyl or —CH(—CH₃)OC(O)-t-butyl, or
—CH₂OC(O)-alkyl, preferably —CH₂OC(O)(—C₁-C₆-alkyl), particularly preferably —CH₂OC(O)-methyl, —CH₂OC(O)-ethyl, —CH₂OC(O)-n-propyl, —CH₂OC(O)-n-butyl or —CH₂OC(O)-t-butyl.

By a group that can be converted in-vivo into a sulphonamide or amino group is meant for example one of the following groups:

—OH, -formyl, —C(O)-alkyl, —C(O)-aryl, —C(O)-heteroaryl, —CH₂OC(O)-alkyl,
—CH(-alkyl)OC(O)-alkyl, —C(-alkyl)(-alkyl)OC(O)-alkyl, —CO₂-alkyl, preferably C₁-C₉-alkoxy-carbonyl, particularly preferably methoxy-carbonyl, ethoxycarbonyl, n-propyloxycarbonyl, isopropyloxycarbonyl, n-butyloxycarbonyl, n-pentyloxycarbonyl, n-hexyloxycarbonyl, cyclohexyloxycarbonyl, n-heptyloxycarbonyl, n-octyloxycarbonyl or n-nonyloxycarbonyl,
—CO₂(—C₁-C₃-alkyl)-aryl, preferably —CO₂(—C₁-C₃-alkyl)-phenyl, particularly preferably benzyloxycarbonyl,
—C(O)-aryl, preferably benzoyl,
—C(O)-heteroaryl, preferably pyridinoyl or nicotinoyl or
—C(O)-alkyl, preferably —C(O)(—C₁-C₆-alkyl), particularly preferably 2-methylsulphonyl-ethoxycarbonyl, 2-(2-ethoxy)-ethoxycarbonyl.

The term halogen generally denotes fluorine, chlorine, bromine or iodine, preferably chlorine or fluorine, particularly preferably fluorine.

The compounds according to the invention may be in the form of the individual optical isomers, mixtures of the individual enantiomers, diastereomers or racemates, prodrugs, double prodrugs and in the form of the tautomers, salts, solvates and hydrates thereof as well as in the form of the free bases or the corresponding acid addition salts with pharmacologically acceptable acids—such as for example acid addition salts with hydrohalic acids, for example hydrochloric or hydrobromic acid, or organic acids, such as for example oxalic acid, fumaric acid, diglycolic acid, formic acid, malic acid, benzoic acid, benzenesulphonic acid, camphorsulphonic acid, acetic acid, ethanesulphonic acid, glutamic acid, maleic acid, mandelic acid, lactic acid, phosphoric acid, nitric acid, sulphuric acid, succinic acid, para-toluenesulphonic acid, trifluoroacetic acid, tartaric acid, citric acid or methanesulphonic acid.

Moreover, if the new compounds of formula I thus obtained contain a carboxy group or another acid group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

Moreover the compounds of general formula I obtained may be resolved into their enantiomers and/or diastereomers.

Thus, for example, the compounds of general formula I obtained which occur as racemates may be separated by methods known per se (cf. Allinger N. L. And Eliel E. L. In “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be, for example, (+) or (−)-menthol and an optically active acyl group in amides, for example, may be a (+)- or (−)-menthyloxycarbonyl.

As has been found, the compounds of general formula (I) are characterised by their great versatility in the therapeutic field. Particular mention should be made of those applications in which the effects of beta-3-agonists, particularly selective beta-3-agonists play a part.

Such diseases include for example:

atherosclerosis, cholangitis, gall bladder disease, chronic cystitis, chronic bladder inflammation; chronic prostatitis, cystospasm, depression, duodenal ulcer, duodenitis, dysmenorrhoea, increased intraocular pressure and glaucoma, enteritis, oesophagitis, gastric ulcer, gastritis, gastrointestinal disorders caused by contraction(s) of the smooth muscle, gastrointestinal disorders incl. gastric ulcer, gastrointestinal ulceration, gastrointestinal ulcers, glaucoma, glucosuria, hyperanakinesia, hypercholesterolaemia, hyperglycaemia, hyperlipaemia, arterial hypertension, hypertriglyceridaemia, insulin resistance, intestinal ulceration or small bowel ulcers (incl. Inflammatory bowel diseases, ulcerative colitis, Crohn's disease and proctitis=inflammation of the rectum), irritable colon and other diseases with decreased intestinal motility, depression, melancholy, pollacisuria, frequent urinary urgency, nervous neurogenic inflammation, neurogenic bladder dysfunction, neurogenic inflammation of the respiratory tract, neuropathic bladder dysfunction, nycturia, non-specific diarrhoea, dumping syndrome, obesity, fatness, pancreatitis, inflammation of the pancreas, stomach ulcers, prostate diseases such as benign prostatic hyperplasia, enlarged prostate, spasm, cramp, type 2 diabetes mellitus, irritable bladder or concrement of the lower urinary tract.

The following may also be mentioned: urge incontinence, stress incontinence, mixed incontinence, overactive bladder (OAB) in the forms of wet OAB or dry OAB, OAB with imperative need to urinate, with or without urge incontinence, with or without increased frequency of urination, with or without nocturnal urination, dysuria, nycturia, pollacisuria, build-up of residual urine. Of these indications, OAB with increased frequency of urination, with or without urge incontinence, with or without nocturnal urination, is preferred.

The compounds may also be used in cases of pain in the prostate or of the lower urogenital tract. The diseases in question include benign prostatic hyperplasiam (BPH), prostatitis, particularly chronic abacterial prostatitis, of neurogenic, muscular or bacterial origin, chronic pain syndrome of the pelvis, pelvic myoneuropathy, prostatodynia, LUTS (lower urinary tract symptoms), obstructive bladder emptying disorders (BOO) and/or prostatopathy.

The use according to the invention is directed not only to causative treatment of the above indications, but also to the treatment of the accompanying symptoms, particularly any related pain or problems of urine release, pain and discomfort in the region of the prostate or the lower urinary tract including the penis, pain during erection or ejaculation, pain on defecation, erectile disorders.

The beta-3 agonists according to the invention are particularly suitable for the treatment of obesity, insulin resistance, type 2 diabetes mellitus, urinary incontinence, irritable colon and other diseases with decreased intestinal motility or depression, particularly for the treatment of diabetes and obesity.

The activity of the beta-3 agonists can be determined for example in a lipolysis test. The test procedure may be carried out as follows:

Adipocytes were isolated from fatty tissue ex vivo by modifying a method according to Rodbell (Rodbell, M. Metabolism of isolated fat cells. I. Effects of hormones on glucose metabolism and lipolysis. J Biol Chem 239: 375-380.1964). The excised fatty tissue was cut into small pieces and mixed with 1 mg/ml collagenase in Krebs Ringer Buffer (KRB) containing 6 mM glucose and 2% albumin by gently shaking for 30-40 min at 37° C. The cells were filtered through a gauze, washed twice with KRB and in each case 50-150 g were centrifuged for 5 min. 10 μl of the centrifuged adipocytes were incubated with 90 μl of a compound according to the invention (agonist) at concentrations of between 10⁻¹⁵ to 10⁻⁴ M. The agonists were incubated over 40 min at 37° C. A varying release of glycerol into the medium indicated that the fat cell lipolysis had altered as a result of the addition of the agonist. Released glycerol was detected enzymatically with a Sigma kit (Triglyceride (GPO Trinder) Reagent A; Cat. # 337-40A), as described below.

Glycerol is phosphorylated by ATP via glycerol kinase. The resulting glycerol-1-phosphate is oxidised by glycerolphosphate oxidase to form dihydroxyacetone phosphate and hydrogen peroxide. Then a quinonimine dye is produced by the peroxidase-catalysed coupling of sodium-N-ethyl-N-(3-sulphopropyl)m-ansidine and 4-aminoantipyrine. The dye has an absorption peak at 540 nm. The absorption is directly proportional to the glycerol concentration in the samples.

The new compounds may be used for the prevention or short-term or long-term treatment of the above-mentioned diseases, and may also be used in conjunction with other active substances used for the same indications. These include, for example, antidiabetics, such as metformin, sulphonylureas (e.g. glibenclamid, tolbutamide, glimepiride), nateglinide, repaglinide, thiazolidinedione (e.g. rosiglitazone, pioglitazone), PPAR-gamma agonists (e.g. GI 262570), alpha-glucosidase inhibitors (e.g. acarbose, voglibose), alpha2 antagonists, insulin and insulin analogues, GLP-1 and GLP-1 analogues (e.g. exendin-4) or amylin. Also, inhibitors of protein tyrosine phosphatase 1, substances which influence deregulated glucose production in the liver, such as e.g. inhibitors of glucose-6-phosphatase, or fructose-1,6-bisphosphatase, glycogen phosphorylase, glucagon receptor antagonists and inhibitors of phosphoenol pyruvate carboxykinase, glycogen synthase kinase or pyruvate dehydrokinase, lipid lowering agents, such as HMG-CoA-reductase inhibitors (e.g. simvastatin, atorvastatin), fibrates (e.g. bezafibrate, fenofibrate), nicotinic acid and its derivatives, cholesterol absorption inhibitors such as for example ezetimibe, bile acid-binding substances such as for example cholestyramine, HDL-raising compounds such as for example inhibitors of CETP or regulators of ABC1 or active substances for the treatment of obesity, such as e.g. sibutramine or tetrahydrolipostatin.

In particular, they may also be combined with drugs for treating high blood pressure such as e.g. all antagonists or ACE inhibitors, diuretics, β-blockers, and other modulators of the adrenergic system or combinations thereof. In addition, combinations with stimulators of the adrenergic system via alpha 1 and alpha 2 and also beta 1, beta 2 and beta 3 receptors are particularly suitable.

The compounds of general formula (I) may be used on their own or in conjunction with other active substances according to the invention, optionally also in conjunction with other pharmacologically active substances. Suitable preparations include for example tablets, capsules, suppositories, solutions, particularly solutions for injection (s.c., i.v., i.m.) and infusion, elixirs, emulsions or dispersible powders. The content of the pharmaceutically active compound(s) should be in the range from 0.1 to 90 wt. %, preferably 0.5 to 50 wt. % of the composition as a whole, i.e. in amounts which are sufficient to achieve the dosage range specified below. The specified doses may be taken several times a day, if necessary.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, optionally organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, preferably oral. For oral administration the tablets may, of course contain, apart from the above-mentioned carriers, additives such as sodium citrate, calcium carbonate and dicalcium phosphate together with various added substances such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation Examples which follow illustrate the present invention without restricting its scope:

### Examples of Pharmaceutical Formulations

| A) | Tablets | per tablet |
|---|---|---|
| | active substance | 100 mg |
| | lactose | 140 mg |
| | corn starch | 240 mg |
| | polyvinylpyrrolidone | 15 mg |
| | magnesium stearate | 5 mg |
| | | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) | Tablets | per tablet |
|---|---|---|
| | active substance | 80 mg |
| | lactose | 55 mg |
| | corn starch | 190 mg |
| | microcrystalline cellulose | 35 mg |
| | polyvinylpyrrolidone | 15 mg |
| | sodium-carboxymethyl starch | 23 mg |
| | magnesium stearate | 2 mg |
| | | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| C) | Ampoule solution | | |
|---|---|---|---|
| | active substance | 50 | mg |
| | sodium chloride | 50 | mg |
| | water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

The following Examples illustrate the present invention without restricting its scope:

Abbreviations used:

### DMF N,N-dimethylformamide

DMPU 1,3-dimethyl-3,4,5,6-tetrahydro-2(1H)-pyrimidinone
NMP 1-methyl-2-pyrrolidone
TFA trifluoroacetic acid
THF tetrahydrofuran

### HPLC Methods:

Retention times were determined using a Type 1100 apparatus made by Agilent (quaternary pump, diode array detector, LC-MSD).

For Methods 1 and 2 a Merck Cromolith Speed ROD column (RP18e, 50×4.6 mm) was used and elution was carried out with mixtures of acetonitrile and water, modified in each case with 0.1% formic acid, at a flow rate of 1.5 ml/min with the following gradient patterns.

| | |
|---|---|
| time [min] | vol % acetonitrile |

Method 1

| | |
|---|---|
| 0.0 | 10 |
| 4.5 | 90 |
| 5.0 | 90 |
| 5.5 | 10 |

Method 2

| | |
|---|---|
| 0.00 | 5 |
| 0.75 | 5 |
| 5.25 | 98 |
| 5.75 | 98 |
| 6.05 | 5 |
| 6.55 | 5 |

### Preparation of the End Products:

### EXAMPLE 1

### [4-(1-{3-[2-(3-phenylsulphonylamino-phenyl)-2-hydroxy-ethylamino]-3-methyl-butyl}-1H-imidazol-4-yl)-phenyl]-acetic acid-hydrotrifluoroacetate

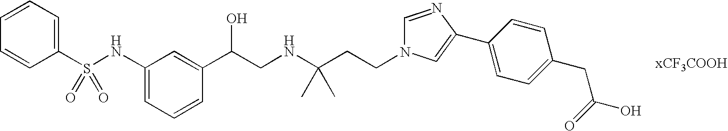

### a. Tert-butyl [3-(4-iodo-imidazol-1-yl)-1,1-dimethyl-propyl]-carbamate

3.88 g (20 mmol) 4-iodoimidazole are dissolved in 30 ml DMPU and at 5° C., 556 mg (22 mmol) 95% sodium hydride are added batchwise. After the development of gas has died down the reaction mixture is stirred for 1 hour at 10° C. Then a solution of 4.44 g (20 mmol) tert-butyl (3-chloro-1,1-dimethyl-propyl)-carbamate in 5 ml DMPU and 739 mg (2.0 mmol) tetrabutylammonium iodide are added. The reaction mixture is stirred for 16 hours at ambient temperature and then heated to 80° C. for 24 hours. After cooling to ambient temperature the reaction solution is poured onto a mixture of 500 ml ice water and 250 ml of ethyl acetate. The aqueous phase is separated off and extracted with ethyl acetate. The combined organic phases are washed with water and saturated aqueous sodium chloride solution, dried on sodium sulphate and evaporated down using the rotary evaporator. The residue is chromatographed on silica gel (petroleum ether/ethyl acetate=80:20→0:100).

Yield: 1.66 g (22% of theory)

C₁₃H₂₂IN₃O₂ (379.24)

Mass spectrum: (M+H)⁺=380

R_{f} value: 0.45 (silica gel; petroleum ether/ethyl acetate=1:1)

### b. 3-(4-iodo-imidazol-1-yl)-1,1-dimethyl-propylamine

1.9 g (5.0 mmol) tert-butyl [3-(4-iodoimidazol-1-yl)-1,1-dimethyl-propyl]-carbamate are dissolved in 90 ml dichloromethane and at ambient temperature 10 ml (130 mmol) TFA are added. The reaction mixture is stirred for 16 hours at ambient temperature and then evaporated down using the rotary evaporator. The residue is taken up in 100 ml 1 N sodium hydroxide solution and 100 ml dichloromethane. The aqueous phase is separated off and extracted with dichloromethane. The combined organic phases are dried on magnesium sulphate and evaporated down using the rotary evaporator.

Yield: 1.37 g (98% of theory)

C₈H₁₄IN₃ (279.12)

Mass spectrum: (M+H)⁺=280

R_{f} value: 0.23 (silica gel; dichloromethane/methanol/NH₄OH=9:1:0.1)

### c. N-[3-(2-ethoxy-2-hydroxyacetyl)-phenyl]-benzenesulphonamide

1 ml of water, 1 g activated charcoal and 2.66 g (24 mmol) selenium dioxide are added to a solution of 1.65 g (6 mmol) N-(3-acetyl-phenyl)-benzenesulphonamide in 10 ml dioxane. The reaction mixture is stirred for 4 days at 80° C. and then evaporated down using the rotary evaporator. The residue is dissolved in 30 ml of ethanol and refluxed for 4 hours. Then the reaction mixture is evaporated down using the rotary evaporator. The residue is dissolved in 100 ml of ethyl acetate, washed several times with 30 ml saturated, aqueous sodium hydrogen carbonate solution, dried on sodium sulphate and evaporated down again using the rotary evaporator. The solid thus obtained is further reacted without any further purification.

Yield of crude product: 917 mg (46% of theory)

C₁₆H₁₇NO₅S (335.38)

### d. N-(3-{1-hydroxy-2-[3-(4-iodimidazol-1-yl)-1,1-dimethyl-propylamino]-ethyl}-phenyl)-benzenesulphonamide

7.81 g (23.3 mmol) N-[3-(2-ethoxy-2-hydroxy-acetyl)-phenyl]-benzenesulphonamide and 6.50 g (23.3 mmol) 3-(4-iodoimidazol-1-yl)-1,1-dimethyl-propylamine are refluxed in 40 ml of ethanol for 15 hours. Then the reaction mixture is cooled to 0° C. and 3.70 g (97.9 mmol) sodium borohydride are added. The mixture is stirred for a further 24 hours at ambient temperature and then 20 ml saturated, aqueous potassium carbonate solution are added. The aqueous phase is separated off and extracted with ethyl acetate. The combined organic phases are washed with 20 ml saturated, aqueous sodium chloride solution, dried on magnesium sulphate and evaporated down using the rotary evaporator. The residue is chromatographed on silica gel (dichloromethane/methanol/NH₄OH=98:2→75:25).

Yield: 5.8 g (45% of theory)

C₂₂H₂₇IN₄O₃S (554.45)

Mass spectrum: (M+H)⁺=555

R_{f} value: 0.34 (silica gel; dichloromethane/methanol/NH₄OH=90:9:1)

### e. [4-(1-{3-[2-(3-phenylsulphonylamino-phenyl)-2-hydroxy-ethylamino]-3-methyl-butyl}-1H-imidazol-4-yl)-phenyl]-acetic acid-hydrotrifluoracetate

100 mg (0.18 mmol) N-(3-{1-hydroxy-2-[3-(4-iodimidazol-1-yl)-1,1-dimethyl-propyl-amino]-ethyl}-phenyl)-benzenesulphonamide are dissolved in 1 ml THF under a protective gas atmosphere and 47.2 mg (0.18 mmol) [4-(4,4,5,5-tetramethyl-1,3,2-dioxaborolan-2-yl)-phenyl]-acetic acid, 2 mg (0.009 mmol) palladium(II)-acetate, 11 mg (0.036 mmol) tri-o-tolylphosphine, 50 mg (0.36 mmol) potassium carbonate are added. The mixture is heated to 160° C. for 10 min in the microwave oven. Then the reaction mixture is added to methanol and neutralised with 4 N hydrochloric acid and freed from the solvent in vacuo. The residue is chromatographed on Varian Microsorb C18-reversed phase [acetonitrile (0.1% TFA)/water (0.13% TFA)=10:90->100:0].

Yield: 16 mg (13% of theory)

C₃₀H₃₄N₄O₅S x C₂HF₃O₂ (676.70)

Mass spectrum: (M+H)⁺=563

retention time HPLC-MS (Method 1): 2.12 minutes

### EXAMPLE 2

### Methyl [4-(1-{3-[2-(3-phenylsulphonylamino-phenyl)-2-hydroxy-ethylamino]-3-methyl-butyl}-1H-imidazol-4-yl)-phenyl]-acetate-hydrotrifluoroacetate

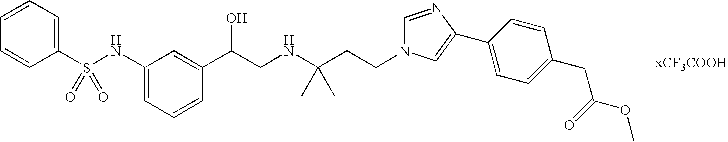

Obtained as a by-product in Example 1e.

Yield: 10 mg (8% of theory)

C₃₁H₃₆N₄O₅S x C₂HF₃O₂ (690.73)

Mass spectrum: (M+H)⁺=577

retention time HPLC-MS (Method 1): 2.36 min

### EXAMPLE 3

### [3-(1-{3-[2-(3-phenylsulphonylamino-phenyl)-2-hydroxy-ethylamino]-3-methyl-butyl}-1H-imidazol-4-yl)-phenyl]-acetic acid-hydrotrifluoroacetate

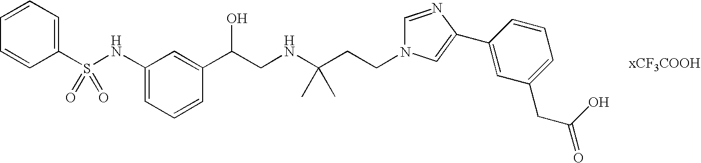

Prepared analogously to Example 1e from N-(3-{1-hydroxy-2-[3-(4-iodimidazol-1-yl)-1,1-dimethyl-propylamino]-ethyl}-phenyl)-benzenesulphonamide and [3-(4,4,5,5-tetramethyl-1,3,2-dioxaborolan-2-yl)-phenyl]-acetic acid.

Yield: 16% of theory

C₃₀H₃₄N₄O₅S x C₂HF₃O₂ (676.70)

Mass spectrum: (M+H)⁺=563

retention time HPLC-MS (Method 1): 2.12 min

### EXAMPLE 4

### [4-(1-{3-[2-(3-phenylsulphonylamino-phenyl)-2-hydroxy-ethylamino]-3-methyl-butyl}-1H-imidazol-4-yl)-phenoxy]-acetic acid-hydrotrifluoroacetate

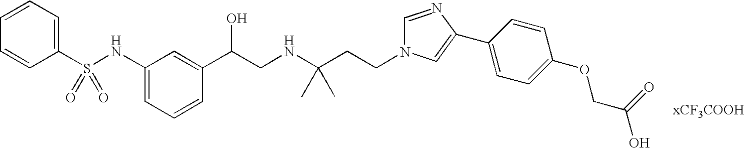

Prepared analogously to Example 1e from N-(3-{1-hydroxy-2-[3-(4-iodimidazol-1-yl)-1,1-dimethyl-propylamino]-ethyl}-phenyl)-benzenesulphonamide and [4-(4,4,5,5-tetramethyl-1,3,2-dioxaborolan-2-yl)-phenoxy]-acetic acid.

Yield: 43% of theory

C₃₀H₃₄N₄O₆S x C₂HF₃O₂ (692.70)

Mass spectrum: (M+H)⁺=579

Retention time HPLC-MS (Method 1): 2.15 min

### EXAMPLE 5

### 3-[4-(1-{3-[2-(3-phenylsulphonylamino-phenyl)-2-hydroxy-ethylamino]-3-methyl-butyl}-1H-imidazol-4-yl)-phenyl]-propionic acid-hydrotrifluoroacetate

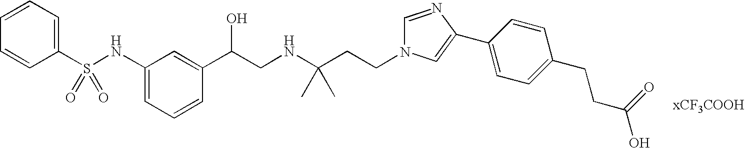

Prepared analogously to Example 1e from N-(3-{1-hydroxy-2-[3-(4-iodimidazol-1-yl)-1,1-dimethyl-propylamino]-ethyl}-phenyl)-benzenesulphonamide and ethyl 3-[4-(4,4,5,5-tetramethyl-1,3,2-dioxaborolan-2-yl)-phenyl]-propionate.

Yield: 26% of theory

C₃₁H₃₆N₄O₅S x C₂HF₃O₂ (690.73)

Mass spectrum: (M+H)⁺=577

retention time HPLC-MS (Method 2): 3.03 min

### EXAMPLE 6

### 3-[3-(1-{3-[2-(3-phenylsulphonylamino-phenyl)-2-hydroxy-ethylamino]-3-methyl-butyl}-1H-imidazol-4-yl)-phenyl]-propionic acid-hydrotrifluoroacetate

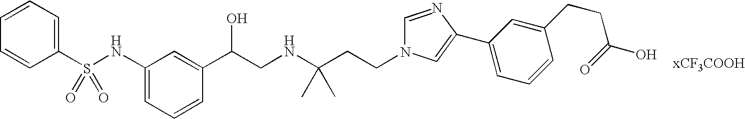

Prepared analogously to Example 1e from N-(3-{1-hydroxy-2-[3-(4-iodimidazol-1-yl)-1,1-dimethyl-propylamino]-ethyl}-phenyl)-benzenesulphonamide and ethyl 3-[3-(4,4,5,5-tetramethyl-1,3,2-dioxaborolan-2-yl)-phenyl]-propionate.

Yield: 22% of theory

C₃₁H₃₆N₄O₅S x C₂HF₃O₂ (690.73)

Mass spectrum: (M+H)⁺=577

Retention time HPLC-MS (Method 2): 3.08 min

### EXAMPLE 7

### Ethyl 3-[3-(1-{3-[2-(3-phenylsulphonylamino-phenyl)-2-hydroxy-ethylamino]-3-methyl-butyl}-1H-imidazol-4-yl)-phenyl]-propionate hydrochloride

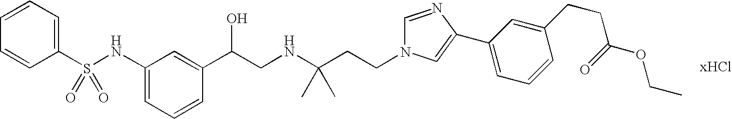

18 mg (0.026 mmol) 3-[3-(1-{3-[2-(3-phenylsulphonylamino-phenyl)-2-hydroxy-ethylamino]-3-methyl-butyl}-1H-imidazol-4-yl)-phenyl]-propionic acid-hydrotrifluoroacetate are heated in 3 ml of ethanolic hydrochloric acid for 10 minutes at 100° C. in the microwave. Then the solvent is eliminated in vacuo.

Yield: 10 mg, 63% of theory

C₃₃H₄₀N₄O₅S x HCl (641.22)

Mass spectrum: (M+H)⁺=605

Retention time HPLC-MS (Method 2): 3.83 min

### EXAMPLE 8

### [3-(1-{3-[2-[3-(phenylsulphonylamino)-phenyl]-2-hydroxy-ethylamino]-3-methyl-butyl}-1H-imidazol-4-yl)-phenoxy]-acetate ethyl-dihydrotrifluoroacetate

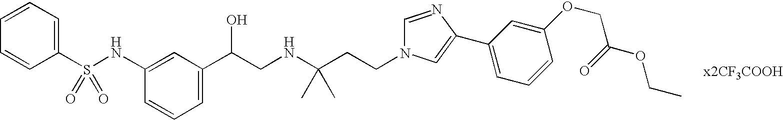

### a. Tert-butyl 4-(3-hydroxy-phenyl)-imidazole-1-carboxylate

400 mg (1.66 mmol) 4-(3-hydroxy-phenyl)-imidazole are suspended in 8 ml THF and 0.7 ml (5 mmol) triethylamine are added. 398 mg (1.83 mmol) di-tert-butyldicarbonate are added and the mixture is stirred for 5 hours at ambient temperature. After the addition of 3 ml THF and 3 ml DMF the mixture is stirred for 16 hours at ambient temperature. The precipitate is suction filtered and washed with THF. The filtrate is freed from the solvent in vacuo.

Yield: 407 mg (94% of theory)

C₁₄H₁₆N₂O₃ (260.29)

Mass spectrum: (M−H)⁻=259

Retention time HPLC-MS (Method 1): 3.29 minutes

### b. methyl [3-(1H-imidazol-4-yl)-phenoxy]-acetate

400 mg (1.54 mmol) tert-butyl 4-(3-hydroxy-phenyl)-imidazole-1-carboxylate are dissolved in 10 ml acetonitrile and 424 mg (3.1 mmol) potassium carbonate are added. Then 0.16 ml (1.7 mmol) methyl bromoacetate are added. The reaction mixture is refluxed for 2 hours. Then the solid is removed by suction filtering and the filtrate is evaporated to dryness. The residue is dissolved in 30 ml of ethanolic hydrochloric acid and stirred for 4 hours at ambient temperature. The solvent is eliminated in vacuo and the residue is chromatographed on Varian Microsorb C18-Reversed phase [acetonitrile (0.1% TFA)/water (0.13% TFA)=10:90->100:0].

Yield: 267 mg (52% of theory)

C₁₂H₁₂N₃O₄ x C₂HF₃O₂ (346.26)

Mass spectrum: (M+H)⁺=233

Retention time HPLC-MS (Method 1): 1.38 minutes

### c. ethyl {3-[1-(3-amino-3-methyl-butyl)-1H-imidazol-4-yl]-phenoxy}-acetate

220 mg (0.95 mmol) methyl [3-(1H-imidazol-4-yl)-phenoxy]-acetate are dissolved in 3 ml DMF and 136 mg (1.2 mmol) potassium-tert-butoxide are added while cooling with ice and stirring. The mixture is stirred for 20 minutes in the cold and then 314 mg tert-butyl 4,4-dimethyl-2,2-dioxo-2-[1,2,3]oxathiazinan-3-carboxylate are added. After 2 hours' stirring at ambient temperature 0.46 ml 1 N hydrochloric acid are added. The reaction mixture is poured onto saturated aqueous ammonium chloride solution. The mixture is extracted with ethyl acetate, the combined organic phases are washed with saturated saline solution, dried on magnesium sulphate and evaporated to dryness. The crude product mixture thus obtained is dissolved in 15 ml THF, combined with 4 ml 1 N sodium hydroxide solution and stirred for 2 hours at 50° C. The solvent is eliminated in vacuo, the residue is taken up in 30 ml of ethanolic hydrochloric acid and refluxed for 2 hours. Then the precipitate is suction filtered and the filtrate is freed from the solvent in vacuo. The residue is divided between THF and saturated potassium carbonate solution. The aqueous phase is extracted with THF. The combined organic phases are washed with saturated sodium chloride solution, dried on magnesium sulphate and evaporated to dryness.

Yield: 234 mg (40% of theory)

C₁₈H₂₅N₃O₃ (331.41)

Mass spectrum: (M+H)⁺=332

Retention time HPLC-MS (Method 1): 1.19 minutes

### d. ethyl [3-(1-{3-[2-[3-(phenylsulphonylamino)-phenyl]-2-hydroxy-ethylamino]-3-methyl-butyl}-1H-imidazol-4-yl)-phenoxy]-acetate dihydrotrifluoroacetate

Prepared analogously to Example 1d by reductive amination of N-[3-(2-ethoxy-2-hydroxy-acetyl)-phenyl]-benzenesulphonamide with ethyl {3-[1-(3-amino-3-methyl-butyl)-1H-imidazol-4-yl]-phenoxy}-acetate and chromatography on Varian Microsorb C18-Reversed phase [acetonitrile (0.1% TFA)/water (0.13% TFA)=10:90->100:0].

Yield: 32% of theory

C₃₂H₃₈N₄O₆S (834.78)

Mass spectrum: (M+H)⁺=607

Retention time HPLC-MS (Method 1): 2.30 minutes

### EXAMPLE 9

### [3-(1-{3-[2-[3-(phenylsulphonylamino)-phenyl]-2-hydroxy-ethylamino]-3-methyl-butyl}-1H-imidazol-4-yl)-phenoxy]-acetic acid

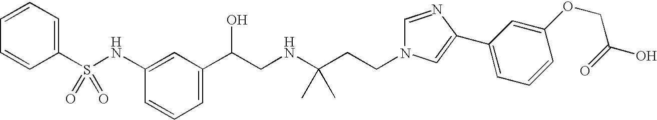

55 mg (0.09 mmol) ethyl [3-(1-{3-[2-[3-(phenylsulphonylamino)-phenyl]-2-hydroxy-ethylamino]-3-methyl-butyl}-1H-imidazol-4-yl)-phenoxy]-acetate dihydrotrifluoroacetate are dissolved in 3 ml THF and 0.4 ml 1 N sodium hydroxide solution are added. The mixture is stirred for 3 h at 50° C., then 0.4 ml of 1 N hydrochloric acid are added and the solvent is eliminated in vacuo.

Yield: 52 mg (98% of theory)

C₃₀H₃₄N₄O₆S (578.68)

Mass spectrum: (M+H)⁺=579

Retention time HPLC-MS (Method 1): 1.96 minutes

### EXAMPLE 10

### 2-[4-(1-{3-[(R)-2-[3-(phenylsulphonylamino)-phenyl]-2-hydroxy-ethylamino]-3-methyl-butyl}-1H-imidazol-4-yl)-phenoxy]-2-methyl-propionic acid-hydrotrifluoroacetate

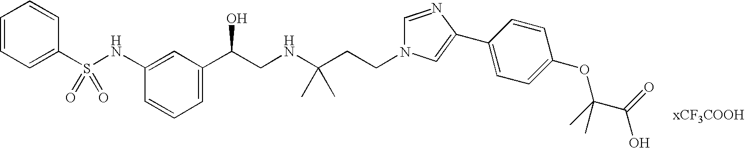

### a. 4-(4-benzyloxy-phenyl)-1H-imidazole

15 g (49 mmol) 1-(4-benzyloxyphenyl)-2-bromoethanone and 39 ml (0.98 mol) formamide are heated to 150° C. for 7 hours. Then ethyl acetate and saturated sodium hydrogen chloride solution are added at ambient temperature. The mixture is extracted with ethyl acetate, the combined organic phases are dried on magnesium sulphate and the solvent is eliminated in vacuo. The residue is triturated with a little acetonitrile, suction filtered and dried.

Yield: 4.7 g (38% of theory)

C₁₆H₁₄N₂O (250.30)

Mass spectrum: (M+H)⁺=251

### b. Tert-butyl {3-[4-(4-benzyloxy-phenyl)-imidazol-1-yl]-1,1-dimethyl-propyl}-carbamate

Prepared analogously to Example 8c from 4-(4-benzyloxy-phenyl)-1H-imidazole and tert-butyl 4,4-dimethyl-2,2-dioxo-2-[1,2,3]oxathiazinan-3-carboxylate with potassium-tert-butoxide in DMF followed by chromatography on silica gel (petroleum ether/ethyl acetate=100:0->40:60).

Yield: 63% of theory

C₂₆H₃₃N₃O₃ (435.56)

Mass spectrum: (M+H)⁺=436

R_{f} value: 0.59 (silica gel; dichloromethane/methanol=95:5)

### c. Tert-butyl {3-[4-(4-hydroxyphenyl)-imidazol-1-yl]-1,1-dimethyl-propyl}-carbamate

1.7 g (3.9 mmol) tert-butyl 3-[4-(4-benzyloxy-phenyl)-imidazol-1-yl]-1,1-dimethyl-propyl}-carbamate are dissolved in 30 ml of methanol, combined with 30 mg palladium catalyst (10% on charcoal) and then hydrogenated for 5 hours at ambient temperature under a hydrogen pressure of 3 bar. Then the reaction mixture is filtered and the filtrate is evaporated down using the rotary evaporator.

Yield: 1.3 g (96% of theory)

C₁₉H₂₇N₃O₃ (345.44)

Mass spectrum: (M+H)⁺=346

R_{f} value: 0.45 (silica gel; dichloromethane/methanol/NH₄OH=90:10:0.1)

### d. ethyl 2-{4-[1-(3-tert-butoxycarbonylamino-3-methyl-butyl)-imidazol-4-yl]-phenoxy}-2-methyl-propionate

1.3 g (3.8 mmol) tert-butyl {3-[4-(4-hydroxy-phenyl)-imidazol-1-yl]-1,1-dimethyl-propyl}-carbamate, 0.67 ml (4.5 mmol) ethyl 2-bromo-2-methylpropionate, 1.2 g (9 mmol) potassium carbonate and 0.56 g (3.78 mmol) sodium iodide are dissolved in 10 ml DMF and stirred for 48 hours at 60° C. Then the reaction mixture is poured into water and extracted with ethyl acetate. The combined organic phases are dried on sodium sulphate and the solvent is eliminated in vacuo. The residue is chromatographed on silica gel (petroleum ether/ethyl acetate=100:0->40:60).

Yield: 170 mg (10% of theory)

C₂₅H₃₇N₃O₅ (459.58)

Mass spectrum: (M+H)⁺=460

R_{f} value: 0.20 (silica gel; petroleum ether/ethyl acetate=1:1)

### e. ethyl 2-{4-[1-(3-amino-3-methyl-butyl)-1H-imidazol-4-yl]-phenoxy}-2-methyl-propionate

Prepared analogously to Example 9c from ethyl 2-{4-[1-(3-tert-butoxycarbonylamino-3-methyl-butyl)-1H-imidazol-4-yl]-phenoxy}-2-methyl-propionate in ethanolic hydrochloric acid.

Yield: 90% of theory

C₂₀H₂₉N₃O₃ (359.47)

Mass spectrum: (M+H)⁺=360

R_{f} value: 0.13 (silica gel; dichloromethane/methanol/NH₄OH=90:10:0.1)

### f. N-(3-acetyl-phenyl)-dibenzenesulphonamide

2.75 g (10 mmol) N-(3-acetyl-phenyl)-benzenesulphonamide are dissolved in 50 ml acetonitrile and 3.3 ml (24 mmol) triethylamine are added. Over a period of 10 minutes at ambient temperature 3.89 g (22 mmol) benzenesulphonic acid chloride are added dropwise with vigorous stirring. The reaction mixture is then stirred for 20 hours at ambient temperature and then evaporated down using the rotary evaporator. The residue is poured into ice water, whereupon a beige solid is precipitated. This precipitate is filtered off and recrystallised from ethyl acetate.

Yield: 3.6 g (87% of theory)

C₂₀H₁₇NO₅S₂ (415.49)

Mass spectrum: (M+NH₄)⁺=433

R_{f}=0.44 (silica gel; toluene/ethyl acetate=9:1)

### g. N-[3-(2-chloro-acetyl)-phenyl]-dibenzenesulphonamide

2.1 ml (26 mmol) sulphuryl chloride are added dropwise over a period of 20 min to 3.6 g (8.66 mmol) N-(3-acetyl-phenyl)-dibenzenesulphonamide in 70 ml DCM and 2.11 ml (52 mmol) methanol at 0° C. with vigorous stirring. The reaction mixture is refluxed for 2.5 hours and then stirred for 18 hours at ambient temperature. Then the reaction solution is washed with water, saturated, aqueous sodium hydrogen carbonate solution and saturated, aqueous sodium chloride solution. The organic phase is separated off, dried on magnesium sulphate and evaporated down using the rotary evaporator. The residue is recrystallised from toluene to form a colourless solid.

Yield: 2.55 g (65% of theory)

C₂₀H₁₆ClNO₅S₂ (449.93)

Mass spectrum: (M+NH₄)⁺=459, 457

R_{f}=0.56 (silica gel; toluene/ethyl acetate=9:1)

### h. N—[(R)-3-oxiranyl-phenyl]-dibenzenesulphonamide

7.84 g (24.4 mmol) (−)-B-chloro-diisopinocampheylboran dissolved in 15 ml THF are added dropwise to a solution of 5.00 g (11.1 mmol) N-[3-(2-chloro-acetyl)-phenyl]-dibenzenesulphonamide in 70 ml THF at −30° C. over a period of 60 minutes. After one hour another 2.00 g (6.24 mmol) (−)-B-chloro-diisopinocampheylboran dissolved in 5 ml THF are added dropwise at −30° C. The mixture is stirred for 14 hours at this temperature and the reaction solution is then poured into a mixture of ice water and saturated sodium hydrogen carbonate solution. It is extracted with ethyl acetate, the combined organic phases are washed and dried on magnesium sulphate. Then the mixture is evaporated to dryness. The residue is chromatographed on silica gel (toluene/ethyl acetate=97.5:2.5→90:10). The intermediate product is triturated with diisopropylether, suction filtered and dried. The solid is dissolved in 30 ml DMF and 8.33 ml 4 N lithiumhydroxid solution are added at −5° C. with stirring within 15 minutes. Meanwhile, to improve the stirrability, 3 ml DMF and 2 ml of water are added. After 25 minutes the reaction mixture is acidified at −5° C. with glacial acetic acid and diluted with water. The solid precipitated is suction filtered, washed several times with ice water and dried. (The product may be obtained in racemic form by reacting N-[3-(2-chloro-acetyl)-phenyl]-dibenzenesulphonamide with borane-THF complex (1M in THF) and then with 4 M lithium hydroxide.)

Yield: 3.65 g (79% of theory)

C₂₀H₁₇NO₅S₂ (415.49)

Mass spectrum: (M+NH₄)⁺=433

R_{f} value: 0.47 (silica gel; toluene/ethyl acetate=9:1)

### i. 2-[4-(1-{3-[(R)-2-[3-(phenylsulphonylamino)-phenyl]-2-hydroxy-ethylamino]-3-methyl-butyl}-1H-imidazol-4-yl)-phenoxy]-2-methyl-propionic acid-hydrotrifluoroacetate

132 mg (0.32 mmol) N—[(R)-3-oxiranyl-phenyl]-dibenzenesulphonamide and 115 mg (0.32 mmol) ethyl 2-{4-[1-(3-amino-3-methyl-butyl)-1H-imidazol-4-yl]-phenoxy}-2-methyl-propionate are heated to 120° C. for 2.5 hours in a melt. Then the mixture is taken up in ethanol and a little dichloromethane at ambient temperature. 1 ml of 4 N sodium hydroxide solution is added and the mixture is stirred for 6 hours at ambient temperature. Then it is acidifed with trifluoroacetic acid. The residue obtained after elimination of the solvent in vacuo is chromatographed on Varian Microsorb C18-Reversed phase [acetonitrile (0.1% TFA)/water (0.13% TFA)=10:90->100:0].

Yield: 80 mg (35% of theory)

C₃₂H₃₈N₄O₆S x C₂HF₃O₂ (720.75)

Mass spectrum: (M+H)⁺=607

Retention time HPLC-MS (Method 1): 2.17 minutes

1: A compound of the formula I 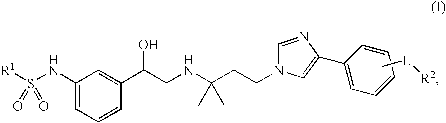
wherein
R¹ denotes a phenyl group which may be substituted by one to three fluorine, chlorine or bromine atoms or one to three C₁₋₃-alkyl, C₁₋₃-alkyloxy, trifluoromethoxy or difluoromethoxy groups, wherein the substituents may be identical or different,
L denotes a C₁₋₃-alkylene group wherein a methylene group may be replaced by an oxygen or sulphur atom or by an NH group, while L may be substituted in the alkyl moiety by one or two methyl groups, and
R² denotes a carboxy or C₁₋₃-alkoxy-carbonyl group,
while the alkyl groups contained in the above-mentioned groups may each be straight-chain or branched,
or a tautomer or salt thereof.

2: A compound of the formula (I) according to claim 1, wherein
R² is defined as in claim 1,
R¹ denotes a phenyl group which may be substituted by a fluorine, chlorine or bromine atom or a C₁₋₃-alkyl, C₁₋₃-alkyloxy, trifluoromethoxy or difluoromethoxy group, and
L denotes a C₁₋₃-alkylene group or a —O—CH₂— group, while L may be substituted in the alkyl moiety by one or two methyl groups,
or a tautomer or salt thereof.

3: A compound of the formula (I) according to claim 2, wherein
R² is defined as in claim 1,
R¹ denotes a phenyl group and
L denotes a —CH₂, —CH₂—CH₂, —O—CH₂ or —O—C(CH₃)₂— group,
or a tautomer or salt thereof.

4: A compound of the formula I according to claim 1 wherein the group -L-R² is in position 3 or 4 of the phenyl ring.

5: A compound of the formula I according to claim 4 wherein the group -L-R² is in the 4 position of the phenyl ring.

6: A compound according to claim 1 wherein the compound is the (R)-enantiomer of formula

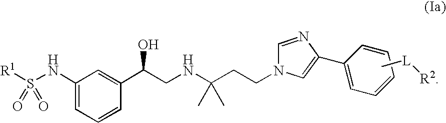

7: A compound of according to claim 1 wherein the compound is the (S)-enantiomer of formula

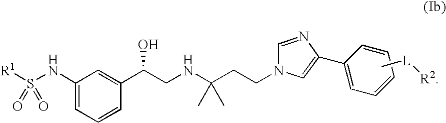

8: A compound according to claim 1 that is selected from the group consisting of:
[4-(1-{3-[2-(3-phenylsulphonylamino-phenyl)-2-hydroxy-ethylamino]-3-methyl-butyl}-1H-imidazol-4-yl)-phenyl]-acetic acid,
methyl [4-(1-{3-[2-(3-phenylsulphonylamino-phenyl)-2-hydroxy-ethylamino]-3-methyl-butyl}-1H-imidazol-4-yl)-phenyl]-acetate,
[3-(1-{3-[2-(3-phenylsulphonylamino-phenyl)-2-hydroxy-ethylamino]-3-methyl-butyl}-1H-imidazol-4-yl)-phenyl]-acetic acid,
[4-(1-{3-[2-(3-phenylsulphonylamino-phenyl)-2-hydroxy-ethylamino]-3-methyl-butyl}-1H-imidazol-4-yl)-phenoxy]-acetic acid,
3-[4-(1-{3-[2-(3-phenylsulphonylamino-phenyl)-2-hydroxy-ethylamino]-3-methyl-butyl}-1H-imidazol-4-yl)-phenyl]-propionic acid,
3-[3-(1-{3-[2-(3-phenylsulphonylamino-phenyl)-2-hydroxy-ethylamino]-3-methyl-butyl}-1H-imidazol-4-yl)-phenyl]-propionic acid,
ethyl 3-[3-(1-{3-[2-(3-phenylsulphonylamino-phenyl)-2-hydroxy-ethylamino]-3-methyl-butyl}-1H-imidazol-4-yl)-phenyl]-propionate,
ethyl [3-(1-{3-[2-[3-(phenylsulphonylamino)-phenyl]-2-hydroxy-ethylamino]-3-methyl-butyl}-1H-imidazol-4-yl)-phenoxy]-acetate,
[3-(1-{3-[2-[3-(phenylsulphonylamino)-phenyl]-2-hydroxy-ethylamino]-3-methyl-butyl}-1H-imidazol-4-yl)-phenoxy]-acetic acid and
2-[4-(1-{3-[(R)-2-[3-(phenylsulphonylamino)-phenyl]-2-hydroxy-ethylamino]-3-methyl-butyl}-1H-imidazol-4-yl)-phenoxy]-2-methyl-propionic acid
or a salt thereof thereof.

9: A physiologically acceptable salt of a compound according to claim 1, 2, 3, 4, 5, 6, 7 or 8.

10-13. (canceled)

14: A pharmaceutical composition comprising a compound according to claim 1, 2, 3, 4, 5, 6, 7 or 8 or a physiologically acceptable salt thereof and a carrier or excipient.

15-16. (canceled)

17: A method for the treatment of obesity or type II diabetes which comprises administering to a host suffering from the same a therapeutically effect amount of a compound according to claim 1, 2, 3, 4, 5, 6, 7 or 8 or a pharmaceutically acceptable salt thereof.
